(19) United States
(12) Patent Application Publication
(10) Pub. No.: US 2004/0166177 A1
(43) Pub. Date: Aug. 26, 2004

(54) Full range nutritional supplements from plant materials and methods for their manufacture

(76) Inventor: Michael Z. Martin, Salinas, CA (US)

Correspondence Address: MORRISON & FOERSTER LLP, 1650 TYSONS BOULEVARD, SUITE 300, MCLEAN, VA, 22102, US

(21) Appl. No.: 10/660,737

(22) Filed: Sep. 12, 2003

Related U.S. Application Data
(60) Provisional application No. 60/409,974, filed on Sep. 12, 2002.

(51) Int. Cl. A61K 35/78

(52) U.S. Cl. 424/725

(57) ABSTRACT

The invention relates to obtaining and concentrating bioactive substances, and to methods for their manufacture from various foodstuffs.

### FIELD OF THE INVENTION

The invention relates to obtaining and concentrating bioactive substances, and to methods for their manufacture from various foodstuffs.

### BACKGROUND OF THE INVENTION

Many plant-derived compounds such as carotenoids, anthocyanins, fatty acids, terpenes, and alkaloids impart important positive pharmacological or “nutraceutical” traits to foods through interactions with cellular processes. For example, carotenoids and anthocyanins exhibit antioxidant activity in cells by maintaining low levels of reactive oxygen intermediates, as anti-inflammatory agents by inhibiting prostaglandin synthesis, or as inhibitors of enzymes involved in cell proliferation. These cellular effects can ameliorate chronic disease such as cancer, arthritis, and cardiovascular disease (Kinsella et al., Food Tech. 85-89 (1993). Accordingly, preparing these compounds for ingestion is a commercially valuable activity. Vibrant dietary supplement/food and nutraceutical industries have evolved to provide these materials that offer significant health benefits to the consumer.

Berries such as blueberry, cranberry, and cherries, as well as other plant stuffs such as grapes and tomatoes are thought to contain specific and numerous bioactive compounds that have multiple beneficial health properties. Even colorants such as anthocyanins have been implicated for their health effects in a wide variety of plantstuffs. For example, anthocyanins such as cyanidin-3-glucoside display strong antioxidant activities (Tsuda, T., et al, J. Agric. Food Chem. 42:2407-2410 (1994)). Such antioxidants may be added to food products to improve shelf life through their actions that block lipid peroxidation. Natural antioxidants may help prevent carcinogenesis. Dietary antioxidants may prevent some peroxidative damage in living systems (Halliwell, B. and J. M. C. Gutteridge, Free radicals in biology and medicine. Oxford University Press, New York 416-494 (1989). Carotenoids also are thought to play an important role in preventing or ameliorating disease processes based on their anti-oxidative activity. Fatty acids, terpenes, and alkaloids represent further groups that have separate and distinct roles in maintaining health.

Methods for extracting these bioactive compounds generally favor one type of bioactive molecule over another. This unfortunately diminishes the value or raises the cost of the product supplied to the consumer. Many methods in the literature for isolating bioactive molecules or groups of molecules originated from basic research on small samples and are not practical for commercial use. Commercial value reflects consumer desires for large quantities of bioactive substances at a reasonable cost. Presently, neutraceuticals are available in mostly rough form and obtained by crude extractions of plant materials that are particularly rich in a desired bioactive compound or group of compounds. Accordingly, the consumer often purchases a product having a composition that is determined by the ease of a particular extraction procedure or availability of a plant having a high concentration of particular compound. Most desired, in contrast, are products that fully exploit the range of bioactivities available in nature, and that comprise different classes of substances that share common effects.

By way of example, Kava contains seven major kavalactones having differential effects in different functions such as an anti-anxiety agent, a short acting topical anaesthetic, a muscle relaxant, and a sleeping aid. A kava extraction or formulation procedure that cannot accommodate a consumer product having desired ratios of the major kavalactones fails to exploit the full opportunity provided by this plant. The lack of control is a particular problem where extraction procedures result in crude, complex formulations that include undesirable bioactive agents with negative unhealthy effects.

An example of a suboptimum extraction procedure is provided in U.S. Pat. No. 5,897,866 issued to Bombardelli et al. on Apr. 27,1999. This document teaches that of the a) aromatic hydrocarbons, b) aliphatic hydrocarbons, and c) halohydrocarbons, hexane and methylene chloride work best for extraction of lycopene from tomato, particularly with a fatty acid additive. This patent describes a multi-step process where lycopene is removed from tomato material by homogenizing, heating and cooling, and centrifugation or filtering, prior to an extraction step with the selected solvent. Alternatively tomato skins are sieved, treated in water for an hour, dried and ground prior to extraction with a hexane solvent. Following extraction and evaporation, a 5% lycopene material was obtained, in a yield of approximately 0.5 gm per 10 kg of starting material. To obtain a more purified material from the lycopene oil, supercritical carbon dioxide was used in a countercurrent three step column, yielding 24 gms of pure product from 500 gm of starting oil. However, a variety of tomatoes contain approximately 100 mg per 100 gm weight (0.1%) (“Lycopene content in Raw Tomato Varieties and Tomato Products by EMAN M. TAWFIK (paper presented at IFT Annual Meeting on the Technical Program Session, Jun. 18, 2002, Anaheim, Calif. USA). Thus the extraction procedure taught in the Bombardelli patent recovers only about 1 percent of the lycopene available and the majority of the lycopene is wasted. Obviously other procedures are needed to commercialize lycopene production from this source.

Similar pretreatment procedures are used for other categories of bioactive substances such as the solvent extraction and chromatography purification of anthocyanins, shown in U.S. Pat. No. 6,423,365 issued Jul. 23, 2002 to Nair. In each case, plant cellular material generally first must be disrupted, a solvent is added to remove the bioactive substance, and then the removed substance is further purified. As mentioned in this patent, unless a very crude material is desired that contains sugars and other undesirable components, an overly expensive step is required to prepare the bioactive substance.

A further problem that has not been addressed sufficiently by this field is the inability to simultaneously extract multiple desired bioactive components from a given plant material. For example, many fruits have desirable quantities of lycopenes and anthocyanins, tannins, condensed tannins (proanthocyanins) and other substances. In order to meet the demand for these materials as neutraceuticals, high efficiency systems are needed that can capture each of these bioactive agents as desired from each plant material. The crude extraction and partial purification procedures known thus far lack both the ability to obtain a suitably wide spectrum of nutrients as well as control the ratios of bioactive compounds, as needed for specific desired effects. Materials with more controllable bioactive compositions and methods for their economical preparation are needed to advance this field and human nutrition generally.

### DESCRIPTION OF THE INVENTION

The above mentioned problems and disadvantages associated with present technology are alleviated by embodiments of the invention. One embodiment provides a soluble composition extracted from a plant material comprising multiple substances within at least 3 classes selected from the group consisting of carotenoids, anthocyanins, fatty acids, terpenes, and alkaloids, wherein the respective weight ratio of substances within each selected class are within 100% of the same ratio for substances in the plant material, and wherein the relative proportion of free sugar in the composition is less than 20% of the free sugar found in the plant material. Another embodiment provides a soluble composition extracted from a plant material comprising organic acids, amino acids, fatty acids, carotenoids, phytosterols, anthocyanins, flavones/isoflavones, saccharides, terpenes, carotenoids, anthocyanins, and complex tannins in relative ratios that are within 100% of their ratios in the unextracted plant material and wherein less than 10% of the free sugar remains.

It has been surprisingly discovered that various classes of chemical compounds can be extracted and concentrated in their full spectrum from a wide variety of foodstuffs. Moreover, the procedures discovered allow fine tuning the chemical profile by adjusting the relative proportions of bioactive substances obtained from a given plant material. This fine tuning control over an extract composition allows formulation of an extract for a targeted pharmaceutical effect. For example, the Kava herb contains a number of kavalactones, and the ratios of, for example, the seven most abundant kavalactones may be optimized in formulations destined for ingestion to acts as an anxiolytic, short-acting topical anesthetic, muscle relaxant, or sleeping aid. The discovered methods both allow the extraction of a wider spectrum of phytochemcial compounds and also the fine tuning of the contents of a given extraction. The methods in many cases provide greater percent recoveries of desired compounds such as lycopene. The methods purify out undesired free sugars, plant water and unsoluble plant solids. Furthermore, many of the methods allow less complicated and less costly extraction with less degradation of desirable phytochemicals, thus, providing compositions of greater potency. A skilled artisan will appreciate additional advantages upon reading the specification and combining the disclosure with general knowledge.

Omit Preliminary Extraction Procedures Used by Others

Most methods known in the art for extracting phytochemicals and preparing neutraceuticals begin with a physical disruption step such as water swelling of plant cells and/or freezing to break open cells, grinding, maceration, or use disrupted plant or fruit mass as starting material. After disruption, fluid is expressed and/or a solvent such as hexane is added to dissolve one or more phytochemicals. Unfortunately, such expression or solvent extraction adds an unwanted step with associated costs and generally removes only a limited or very incomplete spectrum of desired phytochemicals. The incomplete spectrum problem can be addressed by multiple extractions with different solvents, but even then a less than full spectrum of bioactive substances generally is obtained. Embodiments of the invention alleviate these problems by omitting many or most of these steps.

High Pressure Raw Material Chromatography Step

In contrast to the myriad prior art pre-treatments that are used before a chromatography step, it was discovered that much or even all of the initial preparation steps, including, for example, de-pitting, breakage of plant cells, grinding, mashing and the like could be eliminated by a high pressure “raw-material chromatography” step. The term “raw-material chromatography” refers to placing the plant substance into a sealed or sealable chamber and treating the plant substance as a column resin by passing through one or more liquids, gases or supercritical fluids under pressure, to remove a wider spectrum of phytochemicals. Without wishing to be bound by any one theory of how this embodiment of the invention operates, it is believed that the cellulosic component of the plant material acts as a resin support analogously to the resin normally used in chromatography. The solid portion of the plant material surprisingly allows sub-critical gas as well as supercritical gas to permeate the material mass analogously to the way a resin allows permeation in chromatography. Desirably, the cellulosic component is at least 5, 10, 20 or even 30% of the total dried material. In many cases the non-dried raw material may be placed, flowed or pumped into the raw material chromatography chamber for direct high efficiency extraction.

In a desirable embodiment the chamber is round with a height that equals at least one diameter size, and preferably 1.5 to 2.5 times the diameter. Longer columns or chambers of course can be used having lengths that exceed 3, 5, or even 10 times the mean diameter. Cuboidal, irregular and other shapes may be used but for structural and fluidic reasons a cylindrical sized chamber is preferred. The chamber preferably comprises inside surfaces that are resistant to corrosion, and may be made from stainless steel, silicate such as glass, or Teflon. The chamber conveniently will sit upright and fluid or gas or supercritical fluid enters the top, although other arrangements such as horizontal or radial movement in a container in any orientation are suitable. In a desirable embodiment a volume (typically 1 to 2 volumes of the raw material sample volume) of extraction fluid is introduced through a concentric screened rod in the middle of the chamber and extracted fluid (fluid that has passed through the raw-material) is collected at the periphery, through a mesh or other porous surface. In desirable embodiments the raw plant material enters the chamber as a slurry, and may contain solids such as pits or seeds. The extraction may be repeated. In preferred embodiments the extraction is repeated no more than once or twice for a total extracted volume of 3, 4, 5, 6, 8, or less than 10 times the raw material volume.

In an embodiment, whole, partly degraded, ground or crushed natural sources such as plants and/or herbs are placed, flowed, or pumped into the sealable chamber, optionally contacted with a co-solvent and then contacted with a solvent in the liquid phase so as to charge the solvent with analyte. Charged solvent is collected and removed to isolate the analyte. In an embodiment, the herb or plant material contacts the solvent after sealing the chamber and air has been removed. The resulting mixture of solvent and natural source is maintained under pressure so that the natural source and solvent are in intimate contact to charge the solvent with analyte. This type of extraction may be carried out in any vessel that can be sealed and evacuated of air as required. The extraction may be performed at any suitable temperature and is preferably carried out at or below room temperature.

The extracting fluid or gas preferably is introduced at one side or location of the plant raw material column and passes through the column, solvating and picking up phytochemicals that enter the fluid as it traverses the column. The extracted fluid leaves the column space and enters another space where the fluid material is removed, leaving extracted phytochemicals behind as for example described in WO0072861 published Dec. 7, 2000 for ASHRAF-KHORASSANI MEHDI et al. The pressure used within the plant raw material column depends on the type of solvent/gas and the type of phytochemical(s) to be extracted. Preferably the extraction material comprises carbon dioxide, molecular nitrogen (nitrogen gas), hydrogen, an aliphatic or halide carbon compound such as butane, propane, freon, or a mixture such as carbon dioxide with an alcohol, carbon dioxide with ethanol, carbon dioxide with methanol, carbon dioxide with 15% ethanol, and carbon dioxide with alcohol and with isopropyl amine as a secondary modifier. One discovery is that supercritical fluid may be used to remove a wider spectrum of phytochemicals.

Yet another discovery is that a sub-critical pressure may be used to obtain a wide spectrum extract without the higher cost and hazard associated with higher supercritical pressures. Such “sub-critical” pressures generally are between 0.05 and 0.95 times the supercritical pressure for a given temperature, preferably are between 0.25 to 0.8 times and more preferably between 0.5 and 0.7 times the supercritical pressure. For example, the complex tannins from many types of plant materials often are incompletely eluted at low pressures with a single water or water based solvent (water plus water-miscible organic solvent such as an alcohol, or at a high or low pH). It was found that sub critical conditions with carbon dioxide at a pressure between 0.5 and 0.67 times the supercritical pressure for a given temperature often removes more of this group of phytochemicals.

In many cases a single supercritical fluid may be used such as carbon dioxide, propane, butane, isobutane and the like. It was found through experimentation that addition of a small amount of secondary solvent often yields improved extraction. For example, addition of an alcohol such as methanol or ethanol or ethyl ethyl acetate as a cosolvent to, for example a concentration of about 0.02% to 10% (mole ratio) and preferably between 0.1% to 5% of a carbon dioxide solvent can improve recovery of phytochemical.

In another discovery solvents used either at sub-critical pressures or super critical pressures were found to extract phytochemicals from a raw plant material chromatography step. Samples such as whole berries, tomato skins, fruit processing waste, lightly minced herb matter and the like may be treated by the pressure step without any previous processing step. This advantageous feature greatly lowers cost and increases convenience of processing such plant materials into phytochemical extracts. Thus, waste streams may be used directly for low cost high volume extractions.

Another procedure that was discovered and which improves economies of scale, is the direct addition of a co-solvent to the raw material at any time prior to sub critical or super critical chromatography. It was found that a co-solvent such as methanol, ethanol or ethyl acetate may be added at a typical ratio of about 0.1% to 25% of the weight of the sample, and preferably between 0.3% to 5% of the weight of the sample prior to addition of the high pressure solvent. Optionally the sample can be treated with a vacuum after adding the co-solvent and before adding the high pressure solvent.

Another discovery was that nitrogen gas can be used in sub critical conditions with a co-solvent for high efficiency extractions. The co-solvent may be added to the raw material prior to application of high pressure. The co-solvent also may be introduced at the same time as or after the addition of high pressure nitrogen. In one embodiment an alcohol such as methanol, ethanol, propanol or butanol is added and subjected to the high pressure nitrogen, and flowed through the raw material chromatography space. In yet another embodiment the secondary solvent is introduced at an inlet at a separate location and passes through the raw material chromatography space at the high pressure. In another embodiment the secondary solvent is added prior to or after exposing the raw material to a vacuum. The nitrogen gas generally is pressurized to between 100 to 2000 psi, preferably between 300 to 1500 psi, more preferably between 500 to 1200 psi and yet more preferably between 700 to 900 psi. Without wishing to be bound by any one theory for how this embodiment of the invention operates, it is believed that high pressure nitrogen increases the activity of the co-solvent and thereby decreases the amount of co-solvent needed, which lowers solvent costs and improves extraction efficiency.

Yet another discovery was that small changes in pressure used for carbon dioxide sub critical extraction of phytochemicals allows fine control of molecular species solvated and extracted by the high pressure treatment. For example, when extracting kavalactones from kava samples at a constant temperature of less than 30 degress centigrade using high pressure carbon dioxide, altering the pressure by as little as 75, 50, 35, 25,15 or even 10 psi and repeating an extraction can selectively remove a different set of kavalactones. This qualitative procedure is analogous to HPLC in having the ability to separate molecules and has the principle advantage of being easy to scale up to large biomasses. This discovery may be used in other areas of chemistry, particularly analytical chemistry, where a biological sample may be placed into a chamber and high pressure carbon dioxide, preferably with a co-solvent passed through the chamber at different pressures. The eluted material flow stream obtained at different pressures may be detected, for example by absorbance or fluorescence. Comparison of the detected signals with a reference may be carried out to determine the contents or state of the sample. This technique obviates the need for a resin column, as the sample itself becomes the column, and can be applied using nitrogen or other gas instead of or in addition to carbon dioxide.

Low Pressure Aqueous Phase Raw Material Chromatography

Of course, high pressure extraction may be combined with low pressure aqueous extraction method(s). In one embodiment an aqueous phase such as water with up to 40% ethanol or methanol and optionally at up to 60 degrees centigrade is passed through the raw material chromatography space to remove water solutes such as flavonoides. This is followed by high pressure extraction as described above. High pressure extraction under weak solvating conditions (lower pressure and/or temperature) such as liquid carbon dioxide at subcritical conditions removes for example polar compounds such as fatty acids and sterols. If most of the bioactive substances are such compounds then the high pressure extraction preferably occurs without an aqueous extraction step. Organic, less polar substances such as polycyclics preferably can be removed by using conditions that are closer to supercritical, or by switching to supercritical conditions. Accordingly, if different classes of substances need to be removed separately, an aqueous phase extraction may be followed by not only one but two high pressure extractions. For a full spectrum extract at lowest cost, however, it is desirable to subject the original plant material to a simple high pressure extraction.

Even though high pressure raw material chromatography is very good at removing a wide range of substances, it was found that a pre-extraction with low pressure aqueous phase can actually improve recovery from a subsequent high pressure step. For example, it was discovered that tomato polysaccharide is removed more readily after an aqueous carotenoid extraction step from tomato.

Representative Extraction Agents

A wide variety of solvents appropriate for solvating various bioactive substances in natural sources may be used including, but not limited to, alcohols, weak acids, ketones, chloro derivatives, hydrocarbons, fluorinated hydrocarbons, acetates, ethers, or a combination thereof. Due to it's non-flammable nature, as opposed to propane or butane, and excellent solvating properties for a wide range of target analytes, C02 has become the most common volatile substance used in the art of supercritical fluid extraction, and is desirable for many embodiments. However, CO2 in the presence of water can form carbonic acid, which can degrade biomolecules and some metal surfaces used for reaction vessals. Additionally, supercritical CO2 extraction systems often operate at temperatures in excess of 39 C. Holding labile natural materials at such temperatures for long periods during processing may result in thermally or enzymatically induced spoilage. On the other hand significant dissolution was found using sub-critical carbon dioxide conditions as described herein. Non-chlorinated fluorocarbon solvents also can be used, both at sub-critical concentrations and in supercritical conditions. Such solvents as represented by the disclosure of U.S. Pat. No. 5,512,285 are useful for embodiments.

In one embodiment, non-chlorinated fluorocarbon solvents including, but not limited to, trifluoromethane, difluoromethane, fluoromethane, pentafluoroethane, 1,1,1,-trifluoroethane, 1,1-difluoroethane, 1,1,1,2,2,3,3-heptafluoropropane, 1,1,1,3,3,3-hexafluoropropane, 1,1,1,2,2-pentafluoropropane, 2,2,3-hexafluoropropane, 1,1,2,2,3,3-hexafluoropropane, 1,1,1,2,3,3, hexafluoropropane, and 1,1,1,2-tetrafluoroethane may be used. A mixture of these solvents may be used to tailor the boiling point of the mixture to a particular process and facilitate the selective elution of specific bioactive substances. The solvent may be further modified by mixing with another volatile substance such as butane, hexane, ethanol or any other appropriate substance. In a preferred embodiment, the non-fluorocarbon solvent used for extraction is a tetrafluoroethane, preferably 1,1,1,2-tetrafluoroethane. In a further preferred embodiment, the tetrafluoroethane is unmodified.

Representative Extractions

Tomato flakes and cherries were used as representative raw materials and extracted by a variety of solvents and gases at high pressure. In one extraction of cherries, 83% of total carotenoids (mostly lycopene) were removed while eliminating more than 90% of the free sugar. Carbon dioxide supercritical extraction was carried out but was very slow and yielded very little lycopene. Cosolvents were added to the procedure to improve the yield with little success until, surprisingly, high pressure propane without a co-solvent extracted more than 80% of the lycopene.

In other studies, blueberries were placed into a chromatography chamber and extracted first with water and alcohol (10-40% methanol in water) at 100 psi. The aqueous solvent treated material was exposed to a vacuum. Then hydrocarbon (propane) at near supercritical conditions was used by passing 2 to 3 volumes through the column. A wide range of phytoactive substances were removed. The relative proportions of substances in the extract were similar to their proportions in the blueberry plant material.

In another study methanol is injected into cherry material within the chromatography chamber at 3% of the total weight (raw material plus methanol). Then, high pressure propane at 25 degress centigrade is added at a pressure that is 75% of the supercritical pressure. Three volumes of near supercritical propane are passed through and the removed material is dried by decreasing pressure. The extracted material contains carotenoids, anthocyanins, fatty acids, terpenes, and alkaloids in the same relative proportion as that found in the starting material.

In another study kava leaves are extracted directly with carbon dioxide and methanol co-solvent in a chromatography chamber. The pressure used is approximately half of the supercritical pressure. After two volumes are passed through the pressure is increased by 75 psi and two more volumes are passed through. The pressure is increased again by 75 and two more volumes are passed through. This process is repeated 4 more times and the eluates are collected. It is found that different kavalactones are extracted preferentially into the different eluates.

Representative Extractions Obtained

In each study carried out with near super critical or super critical gas conditions the extracted material was soluble. The term “soluble” as used in this context means that the composition does not include plant particulate matter (has less than 1% by weight, preferably less than 0.5%, 0.2%, and even less than 0.1% by weight plant particles) but includes phytochemicals and other molecules that dissolve in at least one solvent near neutral ph (5 to 9), including the supercritical solvent or near supercritical solvent used to prepare the extract. Of course, where two extractions are carried out and combined, all solutes do not necessarily have to dissolve in one common solvent. The extract may be in the form of a powder, liquid or slurry etc. and may be in a form that is easily applied to other foodstuffs or binders. The term “extracted from a plant material” means that the free water in the plant (water not bound to solubles) has been removed.

Many of the extracts prepared according to embodiments of the invention comprise a “full spectrum” of phytochemicals that mimics the spectrum of phytochemicals in the plant material from which they are obtained. The term “mimic” in this context means that the ratios of the phytochemicals in each group (conveniently measured on a weight basis in the dry form) are within 300%, 200%, 100%, 50% and more preferably within 25% of each other. The phycochemical groups may be carotenoids, anthocyanins, fatty acids, terpenes, and alkaloids. Alternatively, the groups may be organic acids, amino acids, fatty acids, carotenoids, phytosterols, anthocyanins, flavones/isoflavones, saccharides, terpenes, carotenoids, anthocyanins, and complex tannins. In many cases only two, three, four, or five members of each group may desirably be compared in this manner, as many plant stuffs tend to have phytochemicals that predominate in one or a few groups only. Generally, then, the comparison is best made by comparing only the groups that contain, taken together, at least 80% of the total phytochemicals of the plant material. For example, if a tomato phytochemical complement is mostly (more than 80%) lycopene, anthocyanins and terpenes, only the carotenoids, anthocyanins and terpenes need to be compared to determine whether a given extract mimics the phytochemical complement of the source plant material.

Some extracts, such as those prepared by differential carbon dioxide pressure treatment described above, will contain different ratios of desired phytochemicals. This provides the artisan the freedom to mix and match different ratios of desired phytochemicals to render a composite product having an innovative and useful set of pharmacological effects, as exemplified above for the kavalactones. This feature allows multi-component formulas in a single dose size. This is particularly made possible by removing the cellulosic and free sugar components of the plant material during the extraction. By further removing unneeded phytochemicals from a particular formulation for a given biological need, dose size may be reduced.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A soluble composition extracted from a plant material comprising multiple substances within at least 3 classes selected from the group consisting of carotenoids, anthocyanins, fatty acids, terpenes, and alkaloids, wherein the respective weight ratio of substances within each selected class are within 100% of the same ratio for substances in the plant material, and wherein the relative proportion of free sugar in the composition is less than 20% of the free sugar found in the plant material.

2. A composition as described in claim 1, wherein at least 4 classes are selected.

3. A composition as described in claim 1, wherein at least 5 classes are selected.

4. A composition as described in claim 1, wherein less than 1% of the original water content of the plant material remains.

5. A composition as described in claim 1, wherein the relative proportion of free sugar in the composition is less than 10% of the free sugar found in the plant material.

6. A composition as described in claim 1, wherein the relative proportion of free sugar in the composition is less than 5% of the free sugar found in the plant material.

7. A soluble composition extracted from a plant material comprising carotenoids, anthocyanins, fatty acids, terpenes, and alkaloids in relative ratios that mimic their ratios in the unextracted plant material and wherein the relative proportion of free sugar is less than 20% of the free sugar in the unextracted plant material.

8. A composition as described in claim 7, wherein less than 1% of the original water content of the plant material remains.

9. A composition as described in claim 7, wherein the relative proportion of free sugar in the composition is less than 10% of the free sugar found in the plant material.

10. A composition as described in claim 7, wherein the relative proportion of free sugar in the composition is less than 5% of the free sugar found in the plant material.

11. A soluble composition extracted from a plant material comprising carotenoids, anthocyanins, and complex tannins in relative ratios that mimic their ratios in the unextracted plant material and wherein less than 10% of the free sugar remains.

12. A soluble composition extracted from a plant material comprising organic acids, amino acids, fatty acids, carotenoids, phytosterols, anthocyanins, flavones/isoflavones, saccharides, terpenes, carotenoids, anthocyanins, and complex tannins in relative ratios that are within 100% of their ratios in the unextracted plant material and wherein less than 10% of the free sugar remains.

13. A composition as described in claim 12, wherein the relative proportion of free sugar is less than 10% of the free sugar found in the plant material.

14. A composition as described in claim 12, wherein the relative proportion of free sugar is less than 5% of the free sugar found in the plant material.
